(19) United States
(12) Patent Application Publication
(10) Pub. No.: US 2010/0190736 A1
(43) Pub. Date: Jul. 29, 2010

(54) MORPHOLINYL ANTHRACYCLINE DERIVATIVE COMBINED WITH PROTEIN KINASE INHIBITORS

(75) Inventors: Maria Cristina Geroni, Milan (IT); Olga Valota, Legnano (IT); Dario Ballinari, San Donato Milanese (IT); Aurelio Marsiglio, Saronno (IT)

Correspondence Address: SCULLY SCOTT MURPHY & PRESSER, PC, 400 GARDEN CITY PLAZA, SUITE 300, GARDEN CITY, NY, 11530, US

(73) Assignee: NERVIANO MEDICAL SCIENCES S.R.L., Nerviano (MI) (IT)

(21) Appl. No.: 12/671,246

(22) PCT Filed: Jul. 23, 2008
(86) PCT No.: PCT/EP08/59621

§ 371 Date: Mar. 9, 2010

(30) Foreign Application Priority Data
Aug. 2, 2007 (EP) 07113731.9

(51) Int. Cl. A61K 31/706; A61P 35/00

(52) U.S. Cl. 514/34

(57) ABSTRACT

The present invention provides the combined use of a morpholinyl anthracycline derivative of formula (I) as defined in the specification or a pharmaceutically acceptable salt thereof, such as nemorubicin hydrochloride, and a protein kinase (PK) inhibitor, in the treatment of tumors. Also provided is the use of the said combinations in the treatment or prevention of metastasis or in the treatment of tumors by inhibition of angiogenesis.

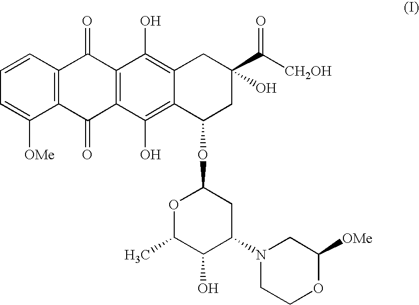

The present invention relates to the field of cancer treatment and provides an antitumor combination comprising a morpholinyl anthracycline derivative and a protein kinase inhibitor, having a synergistic antineoplastic effect. Morpholinyl anthracyclines are known in the art as cytotoxic agents useful in antitumor therapy, see U.S. Pat. No. 4,672,057. Cancers are a leading cause of death in humans; surgery, radiation and chemotherapy are the useful means to fight cancers. In particular, combined chemotherapy, designed to treat cancer by using more than one drug in combination or association, is a well-accepted modality of treatment of neoplastic diseases such as cancer. Several efforts have been and are still being undertaken in order to select antitumor combinations more and more active and safe to be administered to a patient suffering from a cancer. The increase of the antitumor efficacy of a known antitumor compound by administering the same in combination with one or more different antitumor drugs in order to reduce the toxic effects of the individual agents when used alone, and in some instances because the combination has greater efficacy than when either agent is used alone, is a strongly felt need in the field of anticancer therapy.

For example, WO 04/082579 and WO 00/066093 (Nerviano Medical Sciences Srl) are relating to combined use of morpholinyl anthracycline derivatives with radiotherapy or another anticancer drug such as an alkylating agent, an antimetabolite, a topoisomerase I or topoisomerase II inhibitor or a Pt derivative.

The present invention fulfills the need of improved cancer treatment by providing a combined administration of a morpholinyl anthracycline derivative or a pharmaceutically acceptable salt, with a protein kinase inhibitor, having a synergistic antineoplastic effect.

The present invention provides new combinations of a morpholinyl anthracycline derivative with known pharmaceutical agents that are particularly suitable for the treatment of proliferative disorders, especially cancer. More specifically, the combinations of the present invention are very useful in therapy as antitumor agents and lack, in terms of both toxicity and side effects, the drawbacks associated with currently available antitumor drugs.

It is therefore an object of the present invention a combinations comprising a morpholinyl anthracycline derivative having formula (I)

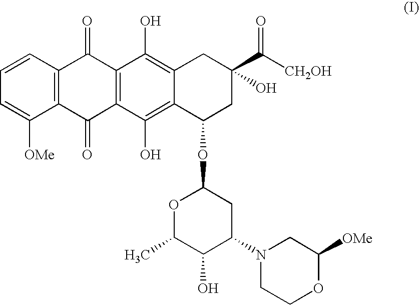

or a pharmaceutically acceptable salt thereof, and a protein kinase inhibitor, having a synergistic antineoplastic effect.

In another aspect, there is provided a pharmaceutical composition comprising a combination according the invention admixed with a pharmaceutically acceptable carrier, diluent or excipient.

A further aspect relates to the use of a combination according the invention for treating a proliferative disorder. A still further aspect relates to a pharmaceutical product comprising a morpholinyl anthracycline as defined above and a protein kinase inhibitor, as a combined preparation for simultaneous, sequential or separate use in therapy. Another aspect relates to a method of treating a proliferative disorder, said method comprising simultaneously, sequentially or separately administering a morpholinyl anthracycline as defined above and a protein kinase inhibitor to a subject. A still further aspect relates to the use of a morpholinyl anthracycline as defined above for the treatment of a proliferative disorder, wherein said treatment comprises simultaneously, sequentially or separately administering a morpholinyl anthracycline as defined above and a protein kinase inhibitor.

Another aspect relates to the use of a morpholinyl anthracycline as defined above and a protein kinase inhibitor for treating a proliferative disorder.

A further aspect of the present invention relates to the use of a combination of a morpholinyl anthracycline derivative having formula (I), as defined above, and a protein kinase inhibitor for the prevention or treatment of metastasis or the treatment of tumors by inhibition of angiogenesis.

In the present description, the morpholinyl anthracycline derivative having formula (I) is nemorubicin, chemical names (8S-cis, 2″S)-7,8,9,10-tetrahydro-6,8,11-trihydroxy-8-(hydroxyacetyl)-1-methoxy-10-{[2,3,6-trideoxy-3-(2-methoxy-4-morpholinyl)-α-L-lyxo-hexopyranosyl]oxy}-5,12-naphthacenedione and 3′ desamino-3′ [2(S)methoxy-4-morpholinyl] doxorubicin.

The term “pharmaceutically acceptable salt” refers to those salts retaining the biological effectiveness and properties of the parent compound. Such salts include acid addition salts obtained by reaction of the free base of the parent compound with inorganic acids such as hydrochloric, hydrobromic, nitric, phosphoric, sulfuric, and perchloric acid and the like; or with organic acids such as acetic, maleic, methanesulphonic, ethanesulfonic, tartaric, citric, succinic and the like.

Preferably, nemorubicin is in the form of its hydrochloride salt.

U.S. Pat. No. 4,672,057 discloses and claims nemorubicin, preparation process, pharmaceutical compositions and medical uses thereof.

In particular, nemorubicin represents a therapeutic option in the treatment of a liver cancer, and nemorubicin administration ways are described and claimed in WO 00/15203 and WO 04/75904.

Protein kinases, hereinafter shortly referred to as PKs, are a large family of homologous proteins [see, for a reference, J. Clin. Invest. 105: 3 (2000); Cancer Chemotherapy and Biological Response Modifiers, Annual 19 Chapter 11, 236 (2001); J. Pharmacol. Exp. Ther. 315:971 (2005)].

PKs, as components of signal transduction pathways, play a central role in diverse biological processes such as control of cell growth, apoptosis, angiogenesis, invasiveness, senescence, metabolism, and differentiation. The development of selective PK inhibitors that can block or modulate diseases with defects in these signaling pathways, has been considered as a promising approach for the development of new anticancer drugs. A selection of these agents is shown in Table 1.

TABLE 1
Low molecular weight protein kinases (PK)
inhibitors in clinical development

| Tyrosine Kinase Target | Inhibitor |
|---|---|
| Bcr-Abl; PDGFR; c-Kit | Imatinib mesylate (STI571; Gleevec) |
| | Dasatinib (BMS-354825; Sprycel) |
| | Nilotinib (AMN-107; Tasigna) |
| | Bosutinib (SKI-606) |
| EGFR | Gefitinib (Iressa; ZD-1839) |
| | Erlotinib (Tarceva; OSI-774) |
| | Lapatinib (GW-572016; Tykerb) |
| | Canertinib (PD-183805; CI-1033) |
| | AEE-788 |
| | XL-647 |
| PKC | Enzastaurin (LY-317615) |
| | UCN-01 |
| | Midostaurin (PKC-412; CGP41251) |
| | Safingol |
| | Perifosine (KRX-0401) |
| | CEP 2563 |
| VEGFR | Sunitinib (Sutent; SU-11248) |
| | Vandetanib (ZD-6474; Zactima) |
| | Recentin (AZD-2171) |
| | Pazopanib (GW-786034) |
| | Vatalanib (PTK-787; CGP-79787) |
| | Axitinib (AG-13736) |
| | Lestaurtinib (CEP-701) |
| | Semaxinib (SU5416) |
| | SU-14813 |
| | BIBF-1120 |
| | XL-880 |
| PDGFR | Leflunomide (SU101) |
| B-Raf | Sorafenib (BAY-43-9006; Nexavar) |
| | PLX-4032 and PLX4720° |
| | XL-281 |
| Cyclin-Dependent Kinase | Flavopiridol (Alvocidib) |
| Ks | Seliciclib (CYC-202) |
| | BMS-387032 (SNS-032) |
| | AT-7519 |
| | R-547 |
| | AG-024322 |
| | P-276-00° |
| | ZK-304709 (ZK-CDK) |
| MEK1, 2 | PD-0325901 |
| | ARRY-142886 (AZD-6244) |

It has now been surprisingly found that the antitumor effect of a morpholinyl anthracycline derivative of formula (I) as defined above is greatly enhanced when it is administered in combination with a PK inhibitor. The effect of the combined administration is significantly increased (synergic effect) with respect to the effect obtained administering each drug as single agent.

A composition according to the present invention comprises preferably a morpholinyl anthracycline derivative of formula (I) as defined above and a PK inhibitor as listed in Table 1 above.

According to a more preferred embodiment of the invention, the PK inhibitor in combination with a morpholinyl anthracycline derivative of formula (I) as defined above is imatinib mesylate, dasatinib, nilotinib, bosutinib, gefitinib, erlotinib, lapatinib, canertenib, enzastaurin, midostaurin, safingol, perifosine, sunitinib, vandetanib, recentin, pazopanib, vatalanib, axitinib, lestaurtinib, semaxinib, SU-14813, sorafenib, flavopiridol, seliciclib, BMS-387032, AT-7519, R-547, AG-024322, P-276-00, ZK-304709, PD-0325901 or ARRY-142886.

According to a more preferred embodiment of the invention, the PK inhibitor is sorafenib or sunitinib combined with nemorubicin hydrochloride.

The term “pharmaceutically effective amount” shall mean that amount of a drug or pharmaceutical agent that will elicit the biological or medical response of a tissue, system, animal or human that is being sought by a researcher or clinician. This amount can be a therapeutically effective amount.

The term “therapeutically-effective” is intended to qualify the amount of each agent for use in the combination therapy, which will achieve the goal of improvement in disease severity and the frequency of incidence over treatment of each agent by itself, and/or of amelioration of adverse side effects typically associated with alternative therapies.

The combined preparations according to the present invention would be useful for the treatment of cancer. Preferably, the subject methods and compositions of the present invention may be used for the treatment of neoplasia disorders including benign, metastatic and malignant neoplasias, and also including acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, lipoma, well differentiated carcinoma, and Wilm's tumor.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of cancer. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has cancer, cardiovascular disease, or pain, inflammation and/or any one of the known inflammation-associated disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

The subject pharmaceutical compositions may be administered to a patient in any acceptable manner that is medically acceptable including orally, parenterally or with locoregional therapeutic approaches such as e.g. implants. Parenteral administration includes administering the constituents of the combined preparation by subcutaneous, intramuscular, intradermal, intramammary, intravenous injections and other administrative methods known in the art. Implants include intra arterial implants, for example, an intrahepatic artery implant.

Any of the combinations of a morpholinyl anthracycline derivative having formula (I) as defined above, and a PK inhibitor are intended as fixed combination (we intend as a drug containing fixed amounts of the two ingredients) and for simultaneous, separate, or sequential use,

By the term “synergistic antineoplastic effect”, as used herein, it is meant the inhibition of the growth tumor, preferably the complete regression of the tumor, by administering an effective amount of the combination comprising a morpholinyl anthracycline derivative having formula (I), and a PK inhibitor.

The constituents of the combined preparations according to the invention can be administered to a patient in any acceptable manner that is medically acceptable including orally, parenterally, or with local therapeutic approaches such as, e.g., implants. Oral administration includes administering the constituents of the combined preparation in a suitable oral form such as, e.g., tablets, capsules, lozenges, suspensions, solutions, emulsions, powders, syrups and the like. Parenteral administration includes administering the constituents of the combined preparation by subcutaneous, intravenous or intramuscular injections. Local therapeutic approaches include implants, for example intra-arterial implants.

Typically a morpholinyl anthracycline derivative having formula (I) is administered intravenously, typically a PK inhibitor is administered intravenously or orally. The actual preferred dosage, method, order and time of administration of the constituents of the combined preparations of the invention may vary according to, inter alia, the particular pharmaceutical formulation of a morpholinyl anthracycline derivative having formula (I), being utilized and the particular pharmaceutical formulation of a PK inhibitor being utilized, the particular cancer being treated, the age, condition, sex and extent of the disease treated and can be determined by one of skill in the art.

The dosage regimen must therefore be tailored to the particular of the patient's conditions, response and associate treatments, in a manner, which is conventional for any therapy, and may need to be adjusted in response to changes in conditions and/or in light of other clinical conditions.

As a non limiting example, suitable dosages of the morpholinyl anthracycline derivative of formula (I) may range from about 0.05 mg/m² to about 100 mg/m² of body surface area and, more preferably, from about 0.1 to about 10 mg/m² of body surface area. For the administration of a PK inhibitor, according to the method of the invention, the course of therapy generally employed may be from 1 mg to 5000 mg. More preferably, the course of therapy employed is from about 10 mg to 2000 mg.

When the active constituents of the combined preparation according to the invention are supplied along with a pharmaceutically acceptable carrier or excipient, a pharmaceutical composition is formed. Such pharmaceutical composition constitutes a further embodiment of the invention.

Pharmaceutically acceptable carriers and excipients are chosen such that side effects from the pharmaceutical compound are minimized and the performance of the compound is not cancelled or inhibited to such an extent that treatment is ineffective. Pharmaceutically acceptable carriers or excipients to be utilized in the preparation of a pharmaceutical composition according to the invention are well known to people skilled in the art of formulating compounds in a form of pharmaceutical compositions. For example, “pharmaceutically acceptable carrier” refers to one or more compatible solid or liquid filler, diluent or encapsulating substances which are suitable for administration to mammals including humans. For example, “pharmaceutically acceptable excipient” refers to any inert substance used as a diluent or vehicle for an active substance(s) that is intentionally added to the formulation of a dosage form. The term includes binders, fillers' disintegrants, and lubricants.

Techniques for formulation and administration of drugs can be found in “Remington's Pharmacological Sciences”; Mack Publishing Co., Easton, Pa., latest edition. Pharmaceutical compositions suitable for parenteral administration are formulated in a sterile form. The sterile composition thus may be a sterile solution or suspension in a non-toxic parenterally acceptable diluent or solvent.

The amount of an active ingredient contained in the pharmaceutical composition according to the invention may vary quite widely depending upon many factors such as, for example, the administration route and the vehicle.

As an example, the pharmaceutical compositions of the invention may contain from about 0.05 mg/m² to about 100 mg/m² of body surface area of a morpholinyl anthracycline derivative of formula (I); and from 1 mg to 2000 mg of a PK inhibitor. Pharmaceutical compositions according to the invention are useful in anticancer therapy. The present invention further provides a commercial kit comprising, in a suitable container means, a morpholinyl anthracycline of formula (I), as defined above, and an antibody inhibiting growth factor or its receptor. In a kit according to the invention a morpholinyl anthracycline derivative of formula (I), as defined above, and a PK inhibitor are present within a single container means or within distinct container means.

Another embodiment of the present invention is a commercial kit comprising a pharmaceutical composition as described above.

Kits according to the invention are intended for simultaneous, separate or sequential use in antitumor therapy. Kits according to the invention are intended for use in anticancer therapy.

The synergistic antineoplastic effect of the combined preparations of the present invention is shown, for instance, by the following in vitro test, which is intended to illustrate the present invention without posing any limitation to it.

### EXAMPLE 1

Table 2 reports the results obtained testing in vitro the cytotoxic effect of nemorubicin in combination with sorafenib

Materials and Methods: Exponentially growing human hepatocellular carcinoma (Hep-G2) and human mammary carcinoma (MCF-7) cell lines were seeded and incubated at 37° C. in a humidified 5% CO₂ atmosphere. Drugs were added to the experimental culture, and incubations were carried out at 37° C. for 72 hours in the dark. Scalar doses of nemorubicin and sorafenib were added to the medium 24 hours after seeding. Two treatment schedules were tested: simultaneous administration (both drugs administered to cells for 72 hours) and sequential administration (nemorubicin administered 24 hours before sorafenib. Nemorubicin and sorafenib solutions were prepared immediately before use. At the end of treatment, cell proliferation was determined by a intracellular adenosine triphosphate monitoring system (CellTiterGlo-Promega) using the Envision (PerkinElmer) as reader Inhibitory activity was evaluated comparing treated versus control data using Assay Explorer (MDL) program. The dose inhibiting 50% of cell growth was calculated using sigmoidal interpolation curve. Combination indices (C.I.) were calculated using a computer program for multiple drug effect analysis based on the equation of Chou-Talalay (Adv Enzyme Regul 1984; 22:27-55) for mutually nonexclusive drugs, where a C.I. of <1 indicates a more than additive effect (C.I.: >3 strong antagonism; 1.3-3 antagonism; 1.2-0.8 additivity; 0.8-0.3 synergism; <0.3 strong synergism).
Results: on both human hepatocellular carcinoma and human mammary carcinoma cell lines the administration of nemorubicin in combination with sorafenib resulted in a synergistic antitumor effect.

TABLE 2
In vitro evaluation of the cytotoxic effect
of nemorubicin in combination with sorafenib

| | Drug | | | | |
|---|---|---|---|---|---|
| | Nemorubicin | Sorafenib | | Combination | Effect of |
| Cell line | nM | nM | Schedule | index | combination |
| Hep-G2 | 0.5 | 20,000 | Simultaneous | 0.66 | Synergism |
| human | 0.2 | 8,000 | | 0.72 | Synergism |
| hepatocellular | 0.5 | 20,000 | Sequential | 0.52 | Synergism |
| carcinoma | 0.2 | 8,000 | | 0.71 | Synergism |
| MCF-7 human | 0.5 | 20,000 | Simultaneous | 0.29 | Strong |
| mammary | | | | | Synergism |
| carcinoma | 0.2 | 8,000 | | 0.26 | Strong |
| | | | | | Synergism |

### EXAMPLE 2

Table 3 reports the results obtained testing in vitro the cytotoxic effect of nemorubicin in combination with sunitinib

Materials and Methods: Exponentially growing human hepatocellular carcinoma (Hep-G2) cells were seeded and incubated at 37° C. in a humidified 5% CO₂ atmosphere. Drugs were added to the experimental culture, and incubations were carried out at 37° C. for 72 hours in the dark. Scalar doses of nemorubicin and sunitinib were added to the medium 24 hours after seeding. Two treatment schedules were tested: simultaneous administration (both drugs administered to cells for 72 hours) and sequential administration (nemorubicin administered 24 hours before sunitinib. Nemorubicin and sunitinib solutions were prepared immediately before use. At the end of treatment, cell proliferation was determined by a intracellular adenosine triphosphate monitoring system (CellTiterGlo-Promega) using the Envision (PerkinElmer) as reader Inhibitory activity was evaluated comparing treated versus control data using Assay Explorer (MDL) program. The dose inhibiting 50% of cell growth was calculated using sigmoidal interpolation curve. Combination indices (C.I.) were calculated using a computer program for multiple drug effect analysis based on the equation of Chou-Talalay (Adv Enzyme Regul 1984; 22:27-55) for mutually nonexclusive drugs, where a C.I. of <1 indicates a more than additive effect (C.I.: >3 strong antagonism; 1.3-3 antagonism; 1.2-0.8 additivity; 0.8-0.3 synergism; <0.3 strong synergism).
Results: on human hepatocellular carcinoma cells the administration of nemorubicin in combination with sunitinib resulted in a synergistic antitumor effect.

TABLE 3
In vitro evaluation of the cytotoxic effect
of nemorubicin in combination with sunitinib

| | Drug | | | | |
|---|---|---|---|---|---|
| | Nemorubicin | Sunitinib | | Combination | Effect of |
| Cell line | nM | nM | Schedule | index | combination |
| Hep-G2 | 0.5 | 20,000 | Simultaneous | 0.8 | Synergism |
| human | 0.2 | 8,000 | | 0.4 | Synergism |
| hepatocellular | 0.5 | 20,000 | Sequential | 0.3 | Synergism |
| carcinoma | 0.2 | 8,000 | | 0.4 | Synergism |

1. A combination comprising a morpholinyl anthracycline derivative having formula (I): 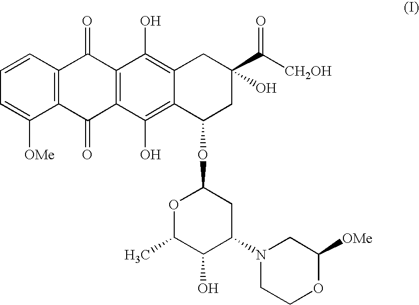
or a pharmaceutically acceptable salt thereof and a protein kinase inhibitor selected from the group consisting of sorafenib and sunitinib, having a synergistic antineoplastic effect.

2. A combination according to claim 1, wherein the morpholinyl anthracycline of formula (I) is nemorubicin hydrochloride.

3. (canceled)

4. (canceled)

5. A pharmaceutical composition comprising a combination as defined in claim 1 admixed with a pharmaceutically acceptable carrier, diluent or excipient.

6. A pharmaceutical product comprising a morpholinyl anthracycline derivative as defined in claim 1 and a protein kinase inhibitor selected from the group consisting of sorafenib and sunitinib, as combined preparation for simultaneous, sequential or separate use in therapy.

7. (canceled)

8. (canceled)

9. A commercial kit comprising, in a suitable container mean, a morpholinyl anthracycline derivative as defined in claim 1, and a protein kinase inhibitor.

10. A commercial kit comprising a pharmaceutical composition or product as defined in claim 9.

11. A kit according to claim 9 for simultaneous, separate or sequential use in antitumor therapy.

12. A method for treating a proliferative disorder comprising simultaneously, sequentially or separately administering a morpholinyl anthracycline derivative as defined in claim 1 and a protein kinase inhibitor.

13. A kit according to claim 10 for simultaneously, separate or sequential use in antitumor therapy.
